(19) United States
(12) Patent Application Publication
(10) Pub. No.: US 2025/0213137 A1
(43) Pub. Date: Jul. 3, 2025

(54) RING ASSEMBLY FOR ANIMAL MONITORING

(71) Applicant: Melissa Brandao, Belgrade, MT (US)

(72) Inventor: Melissa Brandao, Belgrade, MT (US)

(21) Appl. No.: 19/007,194

(22) Filed: Dec. 31, 2024

Related U.S. Application Data
(60) Provisional application No. 63/616,693, filed on Dec. 31, 2023.

(51) Int. Cl. A61B 5/097; A01K 11/00; A01K 15/00; A01K 15/02; A01K 29/00; A61B 5/00; A61B 5/083; G01N 33/497

(52) U.S. Cl. CPC A61B 5/097; A01K 11/008; A01K 15/003; A01K 15/029; A01K 29/005; A61B 5/0008; A61B 5/083; A61B 5/6803; G01N 33/4975; A61B 2503/40

(57) ABSTRACT

A ring assembly has a ring torus shaped body forming an internal sensing cavity with a vent area between the internal cavity and an exterior of the ring torus shaped body. A sensor is positioned at the internal cavity for sensing a level of enteric fermentation methane (or any other substance) emitted by the animal in breath entering the internal sensing cavity via the vent area. An internal circuitry cavity formed by the ring torus shaped body includes a control circuit that is electrically coupled with the methane sensor and further includes a transceiver and a microcontroller programmed to read, at intervals, a methane measurement from the sensor to form methane data and send the methane data via the transceiver to a data service. A ring assembly includes a power source positioned within the internal circuitry cavity for providing power to the control circuit.

### RELATED APPLICATIONS

This application claims priority to U.S. Patent Application Ser. No. 63/616,693, titled “Animal Ring Tag for Methane Measurement in Livestock”, filed Dec. 31, 2023, and incorporated herein by reference in its entirety.

### FIELD

The present application is directed to methane sensing in livestock.

### BACKGROUND

The focus on methane and other greenhouse gases produced by livestock and within livestock operations is driving the need for better methods of methane capture and measurement. Enteric fermentation and the resulting cow burps are one of the leading sources of methane in agriculture. Current solutions for methane detection and/or capture include respiratory chambers, methane sensors at the feed bunk, or sniffers on head harnesses. However, none of these solutions provide for large scale, long-term use, in-field grazing, or continuous real-time data collection.

It is common for domesticated livestock animals, such as cattle, to have be fitted with a device, such as an ear tag or collar, that is machine-interactive to allow location tracking and status tracking status of the animal in a designated setting, such as a dairy farm, feed lot, ranch, etc. The status may include details of animal's health, its behavior, and prove other information, such as on wolf predation, theft, and death.

Livestock are fitted with a nose ring (often called a “bull ring”) to allow the livestock to be led easily by a human. The nose ring is a simple metal or plastic ring that pierces through the nostril, or has an opening that allows it to be slid onto the nose, of the livestock.

### SUMMARY

Estimates of livestock methane output is based on forecasts from fragmentary data collection. There are no viable solutions for continuous and accurate methane measurement of enteric emission in cattle, and therefore data benchmarked by region, breed, feed intake, activity, stage of life and almost any other input variants, is unavailable. However, such information is needed.

Once quantifiable methane outputs are established and benchmarked, methodologies for methane “additionality” in livestock may be improved, verified and registered. Once registered, methane credits may be issued to producers working to reduce methane production in their herds.

Methane detection in livestock is technically challenging. Existing solutions include feed bunks, enclosed chambers, hoods, and sniffers are used to record methane produced from livestock enteric emissions for research or short-term measurements. Optical gas imaging and lasers provide for larger scale (e.g., at the herd level) solutions, but climate, temperature, and windspeeds effect results. Ear tags do not provide a good solution for measuring methane, since they are positioned too far from the animal's mouth and nose. Further, none of these existing solutions collect data continuously or at scale.

Existing data collection and software platform solutions aggregate data from wearable devices and enable a user to perform livestock management tasks more efficiency and with greater precision than when performed by visual checks alone. Animal wearables, like car tags and collars, may indicate a health status and/or a location of the animal. Devices like collars may implement virtual fencing for livestock management. A localized scanning device, such as a small reader, detects signals from a tag or a collar for determining confined animal logistics, such as location, weight, milking, etc. The device (tag or collar) may have one or more LEDs that provide visual aids to animal handlers, such as by indicating a health or a sorting status in feeding or dairy operations.

The embodiments disclosed herein provide improvements over the known art in this field. Certain embodiments are directed to a ring assembly for monitoring and management of animals including, but not limited to, livestock. The ring assembly monitors the animal for one or more of enteric methane emission, temperature, heart-rate, and location. Certain embodiments of the ring assembly may implement a virtual fence that provides an electric shock to the animal when its determined location approaches a defined geographic boundary.

In certain embodiments, the techniques described herein relate to a ring assembly for measurement of methane from an animal, including: a ring torus shaped body forming an internal sensing cavity having a vent area between the internal cavity and an exterior of the ring torus shaped body; a methane sensor positioned at the internal cavity for sensing a methane level in gas entering the internal sensing cavity via the vent area; a control circuit, positioned within an internal circuitry cavity formed by the ring torus shaped body, electrically coupled with the methane sensor, the control circuit having: a transceiver; and a microcontroller programmed to: read, at intervals, a methane measurement from the methane sensor to form methane data; and send the methane data via the transceiver to a data service; and a power source positioned within the internal circuitry cavity for providing power to the control circuit.

In certain embodiments, the techniques described herein relate to a method for determining enteric fermentation methane in breath of an animal, including: positioning, by a ring assembly, a methane sensor proximate a nose of the animal; determining methane data defining a methane level, sensed at intervals, by the methane sensor; and sending the methane data to a data service.

In certain embodiments, the techniques described herein relate to a ring assembly for monitoring an animal, including: a ring torus shaped body forming at least one internal cavity; at least one sensor positioned in the at least one cavity for sensing a status of the animal; and a control circuit, positioned within the at least one cavity, electrically coupled with the at least one sensor and having: a transceiver; and a microcontroller programmed to: read, at intervals, a sensor data from the at least one sensor; and send the sensor data via the transceiver to a data service; and a power source positioned within the at least one cavity for providing power to the control circuit.

### BRIEF DESCRIPTION OF THE FIGURES

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram illustrating one example system for livestock methane measurement using a ring assembly, in embodiments.

FIG. 2 is a schematic diagram illustrating one example closed-ring assembly, in embodiments.

FIG. 3 is a schematic diagram illustrating one example open-ring assembly, in embodiments.

FIG. 4 is a schematic diagram illustrating collection of data by the data service of FIG. 1 from the ring assembly of FIGS. 1, 2, and 3, in embodiments.

FIGS. 5A-5E are schematic diagrams illustrating different views of one example single-use closed-ring assembly that is similar to the closed-ring assembly of FIG. 2, in embodiments.

FIG. 5A is a front view of a closed-ring assembly illustrating vent areas positioned towards a front side of a ring torus shaped body, in embodiments.

FIG. 5B is a rear view of the single-use closed-ring assembly of FIG. 5A illustrating the vent areas, in embodiments.

FIG. 5C is an interior view of the single-use closed-ring assembly of FIG. 5A illustrating positioning of the methane sensors in internal sensing cavities formed by the ring torus shaped body near the vent areas, in embodiments.

FIG. 5D is a front view of the single-use closed-ring assembly of FIG. 5A inserted through a septum of the animal of FIG. 1, in embodiments.

FIG. 5E is a front view of the single-use closed-ring assembly of FIG. 5A prior to closure, in embodiments.

FIGS. 6A-6D are schematic diagrams illustrating different views of one example multi-use open-ring assembly that is similar to the open-ring assembly of FIG. 3, in embodiments.

FIG. 6A is a front facing view of the multi-use ring assembly illustrating positioning of one vent area at a lowest point of ring torus shaped body, in embodiments.

FIG. 6B is a rear view of the open-ring assembly of FIG. 6A, in embodiments.

FIG. 6C is a side view illustrating one example thermal conductive plate positioned on an outer surface of one ring-end of FIG. 6A, in embodiments.

FIG. 6D is a front view of the open-ring assembly of FIG. 6A inserted into a nose of an animal such that the ring-ends contact a septum of the animal, in embodiments.

FIG. 7A is a schematic diagram illustrating one example square-profile open-ring assembly for livestock methane measurement, in embodiments.

FIG. 7B shows a cross-section A-A of the square-profile open-ring assembly of FIG. 7A, in embodiments.

FIG. 8 is a schematic cross-section through the open-ring assembly of FIG. 3 illustrating the sensors in further example detail, in embodiments.

FIG. 9 is a schematic cross-section through the closed-ring assembly of FIG. 2 illustrating the sensor in further example detail, in embodiments.

FIG. 10 is a block diagram illustrating example circuitry that may be used with any of ring assemblies of FIGS. 1, 2, 3, 5A-5E, 6A-6D, and 7A, in embodiments.

FIG. 11 is a flowchart illustrating one example method implemented by the ring assembly of FIG. 1, in embodiments.

FIGS. 12A and 12B illustrate example wireless connectivity of the ring assembly of FIG. 1 with the network, in embodiments.

FIG. 13 is an image showing one example ring assembly fitted to a tail of an animal, in embodiments.

FIG. 14 is an image shown one example ring assembly attached to a leg of a bird, in embodiments.

### DETAILED DESCRIPTION OF THE EMBODIMENTS

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with sensors, computers, processors (hardware processors) memory or other storage have not been shown or described in detail to avoid unnecessarily obscuring descriptions of the various implementations and embodiments.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one implementation” or “an implementation” or “one embodiment” or “an embodiment” means that a particular feature, structure or characteristic described in connection with the embodiment is included in at least one implementation or embodiment. Thus, the appearances of the phrases “one implementation” or “an implementation” or “in one embodiment” or “in an embodiment” in various places throughout this specification are not necessarily all referring to the same implementation or embodiment. Furthermore, the particular features, structures, or characteristics may be combined in any suitable manner in one or more implementations or one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The embodiments described herein are generally directed to systems, methods, and/or apparatus for animal management and/or measurement of methane generated by any type of animal including, but not limited to, livestock in an agricultural setting. However, the disclosed embodiments may be configured to detect any type of gaseous substance and are not limited to sensing methane. The systems, methods and devices may be used with domesticated and wild mammals, including, but not limited to, horses, bison, wolves, large cats, deer, goats, poultry, elephants, and so on. The various techniques disclosed herein may also apply to other animals, including humans.

### Ring Apparatus

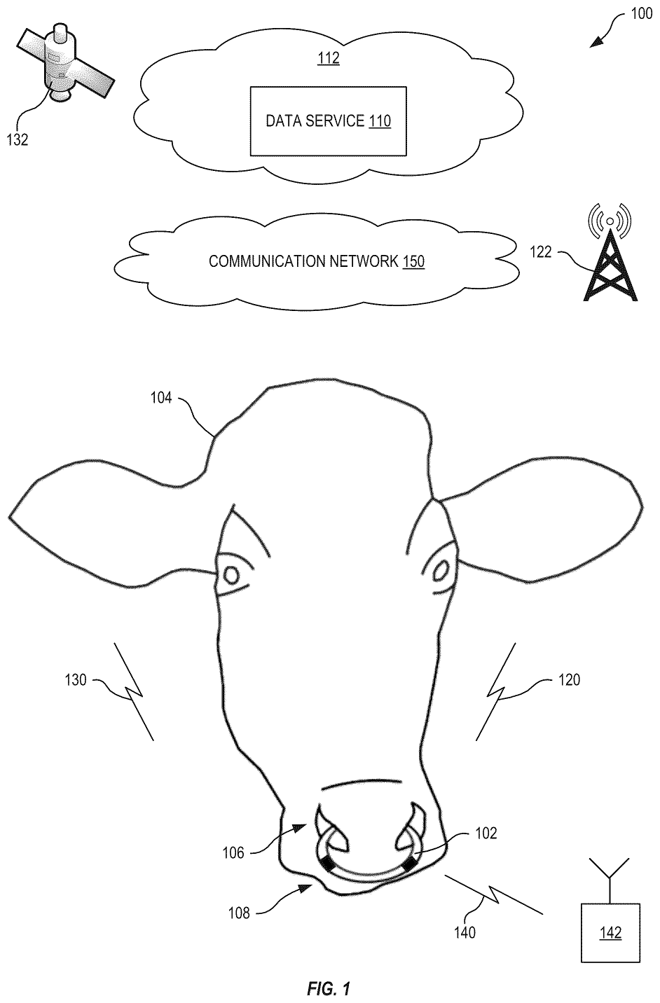

FIG. 1 is a schematic diagram illustrating one example system 100 for livestock methane measurement using a ring assembly 102, in embodiments. Ring assembly 102 attaches to an animal 104 to detect enteric fermentation methane emitted by the animal. Enteric fermentation methane is emitted from a nose 106 and a mouth 108 of animal 104. Ring assembly 102 is similar in size and shape to a nose ring and attaches to nose 106 of animal 104 to be in immediate proximity of gasses exiting nose 106 and mouth 108 (e.g., during breathing, eating, burping and ruminating). Although animal 104 is shown as a cow, animal 104 may represent any type of animal that may be fitted with ring assembly 102, such as horses, bison, wolves, large cats, deer, goats, elephants, chickens, and so on. In one example, a ring assembly may be applied to a leg of a chicken, wherein the ring assembly detects motion and location of the chicken. In another example, a ring assembly is configured with a friction coating (e.g., rubber) that facilitates attachment and retention of the ring assemble at a base of a horses tail, wherein the ring assembly detects levels of methane gas expelled by the horse.

Ring assembly 102 includes sensors for determining a methane level and a transceiver for wirelessly sending the methane level to a data service 110 of system 100. Data service 110 is shown in the cloud 112, but may be implemented elsewhere without departing from the scope hereof. For example, data service 110 may be implemented as a computer server, and may be located in a region of animal 104.

Ring assembly 102 may wirelessly transmit methane data via any available protocol, including as a cellular signal 120 via a cell tower 122 and/or as a satellite signal 130 via a satellite 132—collectively referred to as communication network 150. In certain embodiments, ring assembly 102 transmits methane data using a short-range wireless signal 140 (e.g., using a short-range protocol such as Bluetooth, Wi-Fi, etc.) that is detected by at least one relay device 142 and forwarded to data service 110. Relay device 142 may represent one or more of a bespoke relay device positioned in the vicinity of animal 104, a smart phone, or other such device. In certain embodiments, ring assembly 102 is configured to form a mesh network to relay methane data to data service 110. Accordingly, communication network 150 may include any type of data communication including use of one or more protocols including Bluetooth, Wi-Fi, ANT, LoRa, Internet, cellular, satellite, and any combination thereof. Advantageously, ring assembly 102 monitors animal 102 and thereby determines and reports a status of animal 104.

### Closed-Ring Assembly

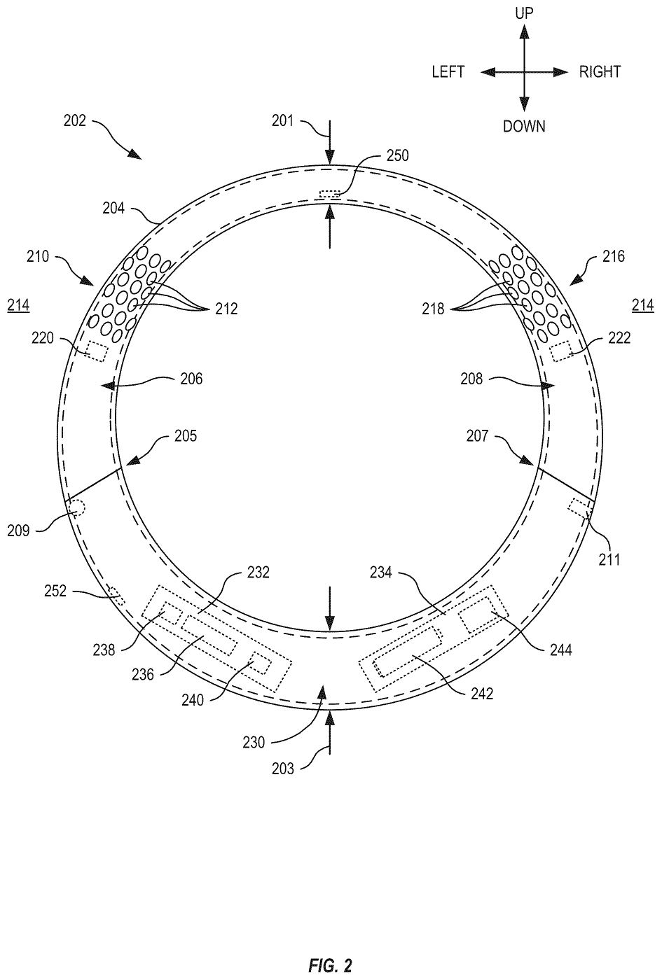

FIG. 2 is a schematic diagram illustrating one example closed-ring assembly 202, in embodiments. Closed-ring assembly 202 may represent ring assembly 102 of FIG. 1 and thus features described above with respect to ring assembly 102 may apply to closed-ring assembly 202 and vice-versa. Closed-ring assembly 202 includes a ring torus shaped body 204 that is hollow and forms an internal sensing cavity 206 and a internal sensing cavity 208. Ring torus shaped body 204 is formed of a substantially rigid material, such as plastic and/or metal. For example, ring torus shaped body 204 may be formed of an injection molded or milled plastic material and/or a molded, forged, and/or formed stainless steel, brass, copper, or aluminum metal. Where tag assembly 202 is made at least in part from an electrically conductive metal, open-ring assembly 202 may have an external insulting coating, such as rubber or plastic.

In certain embodiments, such as where ring torus shaped body 204 is formed of an electrically conductive material (e.g., metal), an external surface of ring torus shaped body 204 is coated by an electrically insulating material such as rubber or plastic.

In certain embodiments, ring torus shaped body 204 is divided, at points 205 and 207, into an upper portion and a lower portion, to facilitate insertion of closed-ring assembly 202 into nose 106 (e.g., through a perforated septum) of animal 104. Ring torus shaped body 204 may include a hinge 209, positioned at point 205 to hingedly couple the upper and lower portions, and further includes a clasp 211 (e.g., a screw or snap mechanism) at point 207 that secures the upper and lower portions together and prevents closed-ring assembly 202 from opening. For example, clasp 211 is opened to allow the upper and lower portions of ring torus shaped body 204 to hinge for insertion and removal of closed-ring assembly 202 from animal 104. In certain embodiments, clasp does not reopen, whereby closed-ring assembly 202 is a single use type.

Internal sensing cavity 206 has a vent area 210 formed by a plurality of apertures 212 in ring torus shaped body 204 between internal sensing cavity 206 and an exterior 214 of ring torus shaped body 204. Similarly, internal sensing cavity 208 has a vent area 216 formed by a plurality of apertures 218 in ring torus shaped body 204 between internal sensing cavity 208 and exterior 214. A methane sensor 220 is positioned at internal sensing cavity 206 and a methane sensor 222 is positioned at internal sensing cavity 208. Vent areas 210 and 216 are positioned to capture enteric emissions from nose 106 and mouth 108 (e.g., during breathing, eating, burping and ruminating) of animal 104 when closed-ring assembly 202 is attached to nose 106 of animal 104. Advantageously, vent areas 210 and 216 are positioned such that they do not accumulate mucus, food or other debris as animal 104 forages or drinks.

As shown in FIG. 2, an upper part of ring torus shaped body 204 has a diameter 201 that is less than a diameter 203 of a lower part of ring torus shaped body 204, thereby giving closed-ring assembly 202 an orientation when hanging from nose 106. Internal sensing cavity 206 is positioned in an upper-left quadrant of ring torus shaped body 204 and internal sensing cavity 208 is positioned in an upper-right quadrant of ring torus shaped body 204. These upper-quadrant positions reduce the likelihood of vent area 210 and vent area 216 being submerged when animal 104 is drinking. However, position of internal sensing cavities 206 and 208 may be at other locations of ring torus shaped body 204 without departing from the scope hereof. For example, based on a type of animal 104, internal sensing cavities 206 and 208 may be positioned in a lower-left quadrant and a lower right quadrant of ring torus shaped body 204. The size and position of vent areas 210 and 216, and size and number of apertures 212, are selected to capture breath from animal 104. In certain embodiments, vent areas 210 and 216 are on a front side of ring torus shaped body 204. In other embodiments, vent areas 210 and 216 are on a front side of ring torus shaped body 204. In other embodiments, vent areas 210 and 216 extend around ring torus shaped body 204.

Ring torus shaped body 204 may have more or fewer internal sensing cavities, vent areas, and sensors without departing from the scope hereof. Closed-ring assembly 202 may also include a sensor 250 for sensing other metrics of animal 104. As shown, sensor 250 is positioned at the top of ring torus shaped body 204, against an inside surface of ring torus shaped body 204, such that it is in close proximity of nose 106 when closed-ring assembly 202 is attached to animal 104. Accordingly, sensor 250 may sense a non-core (e.g., extremity) temperature of animal 104.

Closed-ring assembly 202 may also include an ambient sensor 252, positioned at an external surface of ring torus shaped body 204 and away from contact with animal 104, to sense one or more ambient conditions. For example, ambient sensor 252 may be a temperature sensor for sensing an ambient temperature. In another example, ambient sensor 252 is a light sensor for sensing an ambient light level. Ambient sensor 252 may represent multiple sensors for sensing multiple ambient conditions.

Closed-ring assembly 202 has an internal circuitry cavity 230 for housing a control circuit 232 and a power source 234. Internal circuitry cavity 230 may be fluidly isolated from internal sensing cavities 206 and 208.

### Open-Ring Assembly

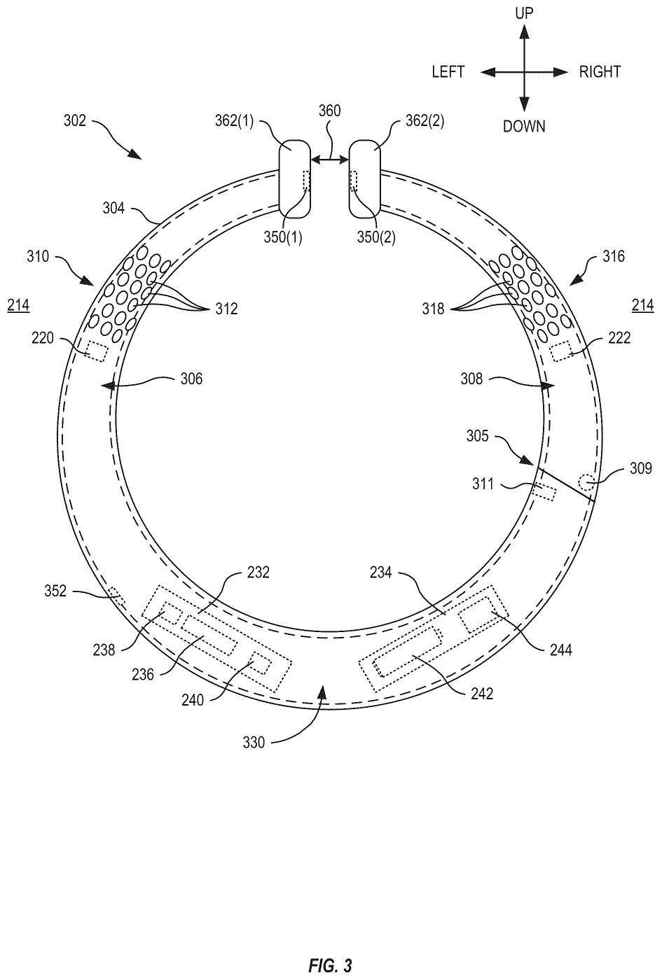

FIG. 3 is a schematic diagram illustrating one example open-ring assembly 302, in embodiments. Open-ring assembly 302 may represent ring assembly 102 of FIG. 1 and thus features described above with respect to ring assembly 102 may apply to open-ring assembly 302 and vice-versa. Open-ring assembly 302 is similar to closed-ring assembly 202 of FIG. 2 and thus features described above with respect to closed-ring assembly 202 (other than the central upper gap 360 discussed below) may apply to open-ring assembly 302 and vice-versa, unless otherwise stated. Open-ring assembly 302 includes a ring torus shaped body 304 that is tapered and hollow to form internal sensing cavities 306 and 308 and an internal circuitry cavity 330. Differing from ring torus shaped body 204, ring torus shaped body 304 forms a central upper gap 360 that is bounded by two rounded-ends 362(1) and 362(2) of ring torus shaped body 304. In the example of FIG. 3, rounded-ends 362 are rounded cylinders or discs; however, rounded-ends 362 may take other forms without departing from the scope hereof. For example, rounded-ends 362 may be substantially spherical in shape (e.g., ball-ends).

Similar to ring torus shaped body 204, ring torus shaped body 304 is formed of a substantially rigid material, such as plastic or metal. For example, ring torus shaped body 304 may be formed of an injection molded or milled plastic material and/or a molded, forged, and/or formed stainless steel, brass, copper, or aluminum metal. Where ring torus shaped body 304 is made at least in part from an electrically conductive metal, ring torus shaped body 304 may have an external insulting coating, such as silicone, rubber, or plastic.

In certain embodiments, ring torus shaped body 304 is divided at a point 305 into two portions that are hingedly attached to one another by a hinge 309 that facilitate insertion of open-ring assembly 302 into nose 106 (e.g., without perforating the septum) of animal 104. Ring torus shaped body 304 further includes a clasp 311 (e.g., a screw or snap mechanism) at point 305 that secures the two portions together and prevents open-ring assembly 302 from opening. For example, clasp 311 is opened to allow the two portions of ring torus shaped body 304 to hinge for insertion and removal of open-ring assembly 302 from animal 104.

Ring torus shaped body 304 forms a plurality of apertures 312 between internal sensing cavity 306 and an exterior 214 to provide a vent area 310 and forms a plurality of apertures 318 between internal sensing cavity 308 and exterior 214 to provide a vent area 316 for internal sensing cavity 308. Advantageously, vent areas 310 and 316 are positioned such that they do not accumulate mucus, food or other debris as animal 104 forages or drinks. As shown in FIG. 3, methane sensors 220 and 222, control circuit 232, and power source 234 may be positioned within ring torus shaped body 304 similarly to positioning in ring torus shaped body 204. Open-ring assembly 302 may include other sensors and functionality without departing from the scope hereof. Open-ring assembly 302 may include on or more sensors 350 for sensing other metrics of animal 104. As shown in FIG. 3, sensors 350(1) and 350(2) are positioned at rounded-ends 362(1) and 362(2), respectively, such that sensors 350 are in close proximity of nose 106 when open-ring assembly 302 is attached to animal 104. Accordingly, sensors 350 may sense a non-core (e.g., extremity) temperature of animal 104.

Open-ring assembly 302 may also include an ambient sensor 352, positioned at an external surface of ring torus shaped body 304 and away from contact with animal 104, to sense one or more ambient conditions. For example, ambient sensor 352 may be a temperature sensor for sensing an ambient temperature. In another example, ambient sensor 352 is a light sensor for sensing an ambient light level. Ambient sensor 352 may represent multiple sensors for sensing multiple ambient conditions.

### Ring Assembly Circuitry

FIGS. 2 and 3 are best viewed together with the following description. Closed-ring assembly 202 and 302 are collectively referred to as ring assemblies 202/302 in the following description.

Control circuit 232 is an electronic circuit that includes at least one microcontroller 236 and a transceiver 238 (e.g., implementing one or more wireless protocols such as satellite, cellular, Bluetooth, etc.). In certain embodiments, transceiver 238 is a transceiver. Microcontroller 236 includes at least one processor and memory storing machine-readable instructions (e.g., firmware). The machine-readable instructions, when executed by the processor, control the processor to implement functionality of ring assembly 202/302 as described herein. Microcontroller 236 may also include one or more electrical interfaces, one or more analog-to-digital converters, and one or more sensors. In certain embodiments, control circuit 232 is implemented as a flex circuit (e.g., components mounted to a flexible circuit board) that allows control circuit 232 to conform to constraints of internal circuitry cavity 230. In certain embodiments, control circuit 232 also includes a Global Navigation Satellite System (GNSS) receiver 240 (e.g., a Global Positioning System-GPS-receiver) for determining a geographic location of ring assembly 202/302. In one example of operation, at intervals, control circuit 232 controls GNSS receiver 240 to determine a current geographic location of ring assembly 202/302 and sends this location to data service 110 via transceiver 238. Control circuit 232 may implement other types of locationing depending on an operational area of ring assembly 202/302. For example, when operating indoors, ring assembly 202/302 may include additional receivers that determine location using an indoor positioning system (IPS).

Power source 234 may include a battery 242 such as one or more of a primary/replaceable battery, a rechargeable battery, a super capacitor, and so on. In certain embodiments, battery 242 is a rechargeable battery and power source 234 also include an energy harvester 244 that harvests energy from the environment (e.g., solar, thermal) and/or movement (e.g., kinetic energy) of ring assembly 202/302 (e.g., as ring assembly 202/302 is moved by animal 104). In certain embodiments, battery 242 is accessible for replacement when ring torus shaped body 204 is open. In other embodiments, ring assembly 202/302 is a single use device and battery 242 is not replaceable. In certain embodiments, energy harvester 244 is configured to magnetically couple with a charging device to recharge battery 242.

Control circuit 232 is electrically coupled (e.g., via part of the flex circuit) with methane sensor 220 and methane sensor 222. Microcontroller 236 is programmed to control, at a first interval, methane sensors 220 and 222 to capture methane levels of gasses within internal sensing cavity 206/306 and internal sensing cavity 208/308, respectively. Accordingly, at the first interval, control circuit 232 determines methane levels of gases entering internal sensing cavity 206/306 and internal sensing cavity 208/308 via vent area 210/310 and vent area 216/316, respectively. At a second interval, which may be the same as, or different from, the first interval, control circuit 232 sends methane data (e.g., the captured methane levels) to data service 110 via transceiver 238. In certain embodiments, microcontroller 236 is further programmed to process captured methane data prior to sending via the transceiver. For example, microcontroller 236 may implement one or more algorithms to pre-process the methane data prior to sending to data service 110. In other embodiments, microcontroller 236 sends raw methane data to data service 110 where it is further processed. Microcontroller 236 may also buffer methane data in its memory, whereby the second interval is longer than the first interval. That is, microcontroller 236 sends the methane data to data service 110 in batches, and not at each reading of methane sensors 220/222. In one example, the first interval is between ten seconds and five minutes and the second interval is between five minutes and five hours. In certain embodiments, the intervals are dynamic and may be changed based on sensed activities and/or events. For example, when animal 104 is resting at night, the intervals may be increased. Similarly, when a methane event or other animal activity is detected, the interval may be reduced. In certain embodiments, these intervals may be set from an external device (e.g., data service 110) via wireless communication.

In certain embodiments, sensors 250/350 are temperature sensors that senses a temperature of animal 104. In another embodiments, sensors 250/350 are ECG probes that senses a pulse of animal 104. Sensors 250/350 may also represent groups of sensors that sense multiple metrics of animal 104. Microcontroller 236 is programmed to read sensor data from sensors 250/350 at a third interval, which may be the same or different from the first interval. For example, microcontroller 236 may read sensors 250/350 at the same interval as methane sensors 220/222 and send the sensor data to data service 110 at the same interval as the methane data. Microcontroller 236 may control GNSS receiver 240 to determine a current location of animal 104 at a fourth interval, between thirty minutes and one hour, and send the location data to 110 with a next transmission of methane data.

Methane sensors 220 and 222 may be of any type of sensor that detects a level of methane in a gas. For example, methane sensors 220 and 222 may be one of non-dispersive infrared (NDIR) sensors, a chemiresistive sensor (e.g., a quantum dot sensors operating on the principle of chemiresistance), and Optical Sensing (e.g., quantum dot sensors using optical methods).

Advantageously, the proximity of ring assemblies 202/302 to nose 106 and mouth 108 of animal 104 allows microcontroller 236 to collect methane data defining methane levels in enteric emissions from the individual animal as a single data point. The single data points captured by ring assembly 202/302 apply specifically to animal 104 to which ring assembly 202/302 is attached. As compared to prior art solutions, where a methane sensor is positioned in a semi-enclosed feed box that captures enteric fermentation methane from any animal using the feed box, and only when the animal's head remains at the feed box, ring assembly 202/302 is positioned at nose 106 of animal 104 and therefore captures methane data of that animal. Since ring assemblies 202/302 remain attached to animal 104, methane data may be collected continuously and independently of the animal's location without undo restriction of the animal's movements. Accordingly, the collected methane data may be analyzed over selected time intervals and/or independently of the animal's activities. The methane data may also be analyzed over one or more defined period, such as one hour, one day, one week, and so on.

Microcontroller 236 may be further programmed to determine a baseline measurement for the methane data that defines a nominal expected methane level for the animal over time. Microcontroller 236 may be further programmed to compare recently obtained methane data against the baseline level for animal 104 to detect significant changes in magnitude of the methane level that may indicate an anomaly for the animal. For example, where methane data indicates current methane levels for animal 104 are greater than a threshold level above the baseline measurement, microcontroller 236 may send a first alert to data service 110 to indicate the anomaly. Similarly, where methane data indicates current methane levels for animal 104 are below a threshold level less than the baseline measurement, microcontroller 236 may send a second alert to data service 110 to indicate the anomaly. The first and second alerts may indicate a change in wellbeing of animal 104. The second alert may also indicate a blockage of vent areas 210/310 and/or 216/316.

Where sensors 250/350 represent temperature sensors for sensing a nostril temperature of animal 104, microcontroller 236 is further programmed to capture a first sequence of temperature measurements from sensors 250/350. Where sensors 252/352 represent ambient temperature sensors, microcontroller 236 is further programmed to capture a second sequence of temperature measurements from sensors 252/352.

In certain embodiments, transceiver 238 is configured to receive the second sequence of ambient temperature measurements from an ambient temperature sensor disposed external to ring assembly 202/302, such as in a nearby data collection unit, from a different tag assembly on another animal, and so on. The first and second sequences of temperature measurements may be correlated to ascertain a state of animal 104, for example. Additional sensors of various types may further provide information regarding the state of the animal.

In some cases, ring assembly 202/302 has a single use configuration where closed-ring assembly 202/302 is permanently attached (e.g., mechanically locked closed) to nose 106 of animal 104, where removal stops operation of ring assembly 202/302 (e.g., breaks ring torus shaped body 204/304 and prevents reuse), and further implements animal and plant health inspection service (APHIS) identification and registration of animal 104.

Ring assemblies 202/302 may be configured to interface with various communication devices locally and/or over one or more networks, such as cellular networks (e.g., via cell tower 122), satellite networks (e.g., via satellite 132), and local relay devices 142 (e.g., Wi-Fi, Bluetooth, etc.), including networks formed by other nearby ring assemblies 202/302. Although shown as a cloud-based service, data service 110 may also be elsewhere without departing from the scope hereof. For example, data service 110 may be implemented within at least one relay device 142, and is thereby local to animal 104. Data service 110 may also be implemented as a remote server connected via one or more of Bluetooth, Wi-Fi, and so on. Data collected from ring assemblies 202/302 may be analyzed to further various livestock management efforts.

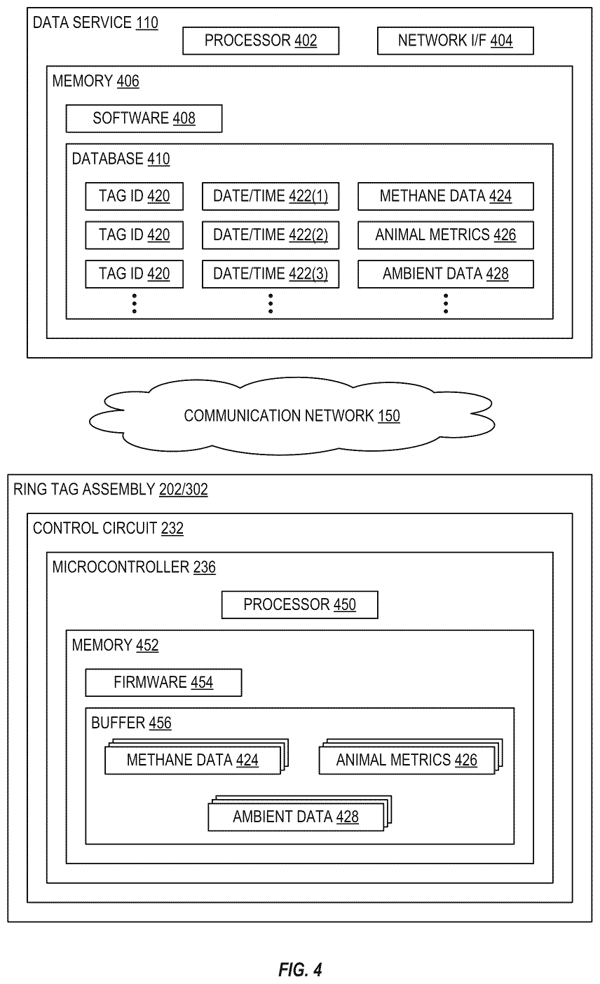

FIG. 4 is a schematic diagram illustrating collection of data by data service 110 of FIG. 1 from ring assembly 102/202/302 of FIGS. 1, 2, and 3, respectively, in embodiments. FIGS. 1-4 are best viewed together with the following description.

Data service 110 includes at least one processor 402, a network interface 404, and memory 406 storing software 408 and a database 410. Software 408 includes machine readable instructions that when executed by processor 402 implement functionality of data service 110, as described herein.

Within ring assembly 102/202/302, microcontroller 236 of control circuit 232 includes at least one processor 450 and memory 452 storing firmware 454. Firmware 454 includes machine readable instructions that when executed by processor 450 cause microcontroller 236 to capture methane data 424 from methane sensors 220/222 at intervals. Firmware 454 may implement a buffer 456 (e.g., a cyclic buffer) in memory 452 to store methane data 424 prior to transmission of methane data 424 to data service 110. Firmware 454 may also cause microcontroller 236 to capture animal metrics 426 from one or more of sensors 250/350 and to capture ambient data 428 from one or more of sensors 252/352. Firmware 454 may also store animal metrics 426 and/or ambient data 428 in buffer 456. At intervals, firmware 454 causes microcontroller 236 to send methane data 424, animal metrics 426, and/or ambient data 428 to data service 110 via transceiver 238.

Software 408 controls network interface 404 to receive methane data 424, animal metrics 426, and/or ambient data 428 from ring assembly 102/202/302, and stores methane data 424, animal metrics 426, and/or ambient data 428 in database 410 in association with a tag ID 420 corresponding to a unique ID of ring assembly 102/202/302 and a date/time 422 (e.g., a time of receipt based on a local real-time clock).

Software 408 may implement one or more algorithms for processing methane data 424, animal metrics 426, and/or ambient data 428 to support management of livestock. In certain embodiments, software 408 determines a health state of animal 104 responsive to a difference between a magnitude of the septum temperature data and a magnitude of a set of ambient temperature data. For example, the ambient temperature data is obtained from ambient sensor 252/352 and/or an ambient temperature sensor proximate animal 104 over a selected time interval. In certain embodiments, software 408 correlates methane measurements received from ring assembly 102 to known or sensed additional information of the animal, such as feed, location, and stage of life.

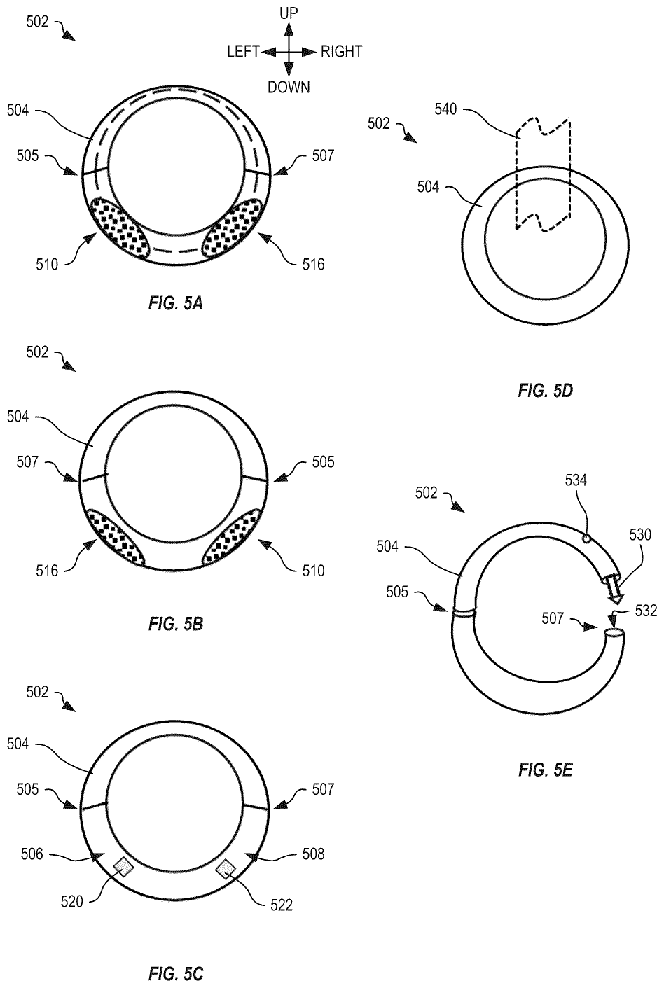

FIGS. 5A-5E are schematic diagrams illustrating different views of one example single-use closed-ring assembly 502 that is similar to closed-ring assembly 202 of FIG. 2, in embodiments. Single-use closed-ring assembly 502 includes two vent areas 510 and 516 that are positioned in lower left and lower right quadrants of a ring torus shaped body 504 of single-use closed-ring assembly 502. Ring torus shaped body 504 may be formed in two part, having a hinge at point 505 and a fastener at point 507. All elements may not be shown in each of FIGS. 5A through 5E for clarity of illustration.

FIG. 5A is a front view of closed-ring assembly 502 illustrating vent areas 510 and 516 positioned towards a front side of ring torus shaped body 504. FIG. 5B is a rear view of single-use closed-ring assembly 502 illustrating vent areas 510 and 516. FIG. 5C is an interior view of single-use closed-ring assembly 502 illustrating positioning of methane sensors 520 and 522 in internal sensing cavities 506 and 508 formed by ring torus shaped body 504 near vent areas 516 and 510, respectively. FIG. 5D is a front view of single-use closed-ring assembly 502 inserted through a septum 540 of animal 104. FIG. 5E is a front view of single-use closed-ring assembly 502 prior to closure, illustrating a single use clasp 530 that is inserted into a non-releasing receptacle 532 to lock single-use closed-ring assembly 502 closed. In certain embodiments, ring torus shaped body 504 may further include a locking mechanism 534 (e.g., screw) that further secures single-use closed-ring assembly 502 closed.

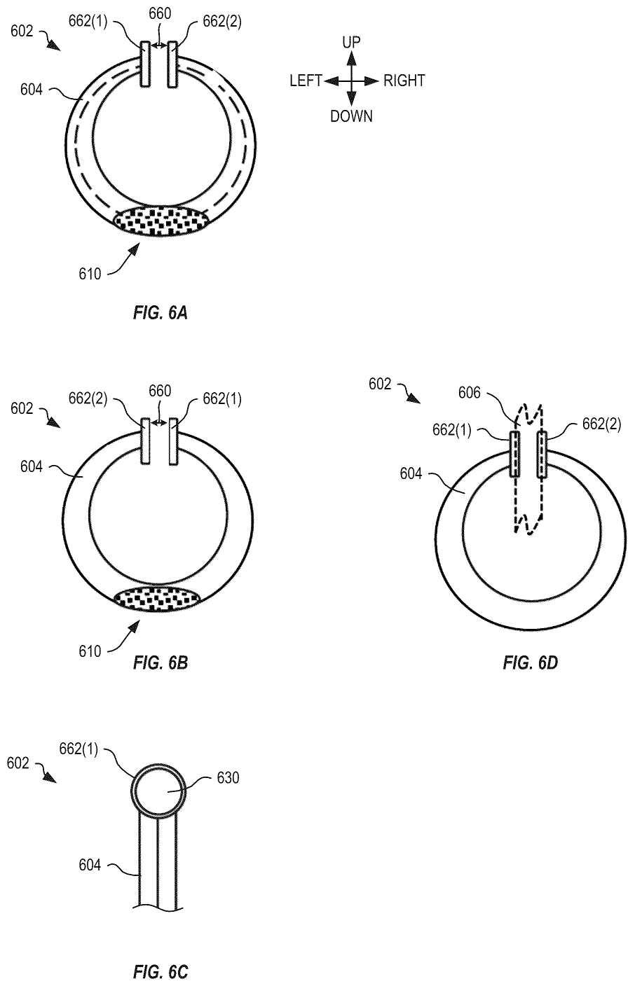

FIGS. 6A-6D are schematic diagrams illustrating different views of one example multi-use open-ring assembly 602 that is similar to open-ring assembly 302 of FIG. 3, in embodiments. Multi-use open-ring assembly 602 includes one vent area 610 that is positioned at a lower mid-portion of a ring torus shaped body 604 of multi-use open-ring assembly 602. Ring torus shaped body 604 may be formed as a single part with a gap 660 formed between two ring-ends 662(1) and 662(2). All elements may not be shown in each of FIGS. 6A through 6D for clarity of illustration.

FIG. 6A is a front facing view of the multi-use ring assembly 602 illustrating positioning of one vent area 610 at a lowest point of ring torus shaped body 604. FIG. 6B is a rear view of open-ring assembly 602. FIG. 6C is a side view of ring-end 662(1) further illustrating one example thermal conductive plate 630 positioned on an outer surface of ring-end 662(1). FIG. 6D is a front view of open-ring assembly 602 inserted into nose 106 of animal 104 such that ring-ends 662(1) and 662(2) to contact a septum 606 of the animal.

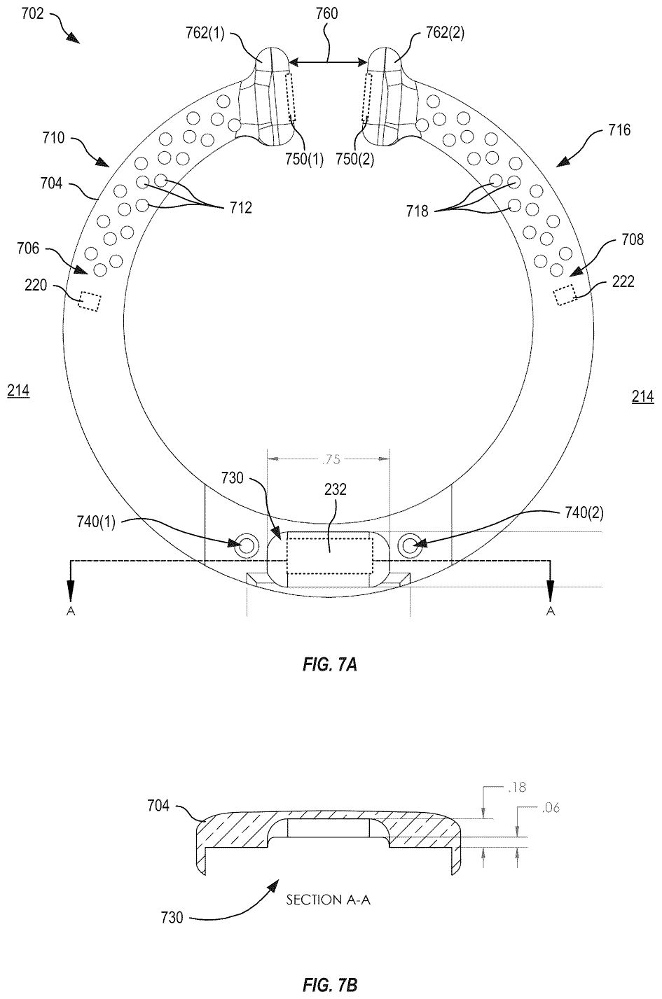

FIG. 7A is a schematic diagram illustrating one example square-profile open-ring assembly 702 for livestock methane measurement, in embodiments. FIG. 7B shows a cross-section A-A of square-profile open-ring assembly 702 of FIG. 7A. FIGS. 7A and 7B are best viewed together with the following description.

Square-profile open-ring assembly 702 is similar to open-ring assembly 302 of FIG. 3, but illustrates an alternative design for access to an internal circuitry cavity 730 that houses control circuit 232. Square-profile open-ring assembly 702 may represent ring assembly 102 of FIG. 1. Square-profile open-ring assembly 702 includes a ring torus shaped body 704 that is tapered and hollow to form internal sensing cavities 706 and 708 and internal circuitry cavity 730. Ring torus shaped body 704 forms a central upper gap 760 that is bounded by two rounded-ends 762(1) and 762(2) of ring torus shaped body 704. In the example of FIG. 7A, rounded-ends 762 are rounded cylinders or discs; however, rounded-ends 762 may take other forms without departing from the scope hereof. For example, rounded-ends 762 may be substantially spherical in shape (e.g., ball-ends).

Similar to ring torus shaped body 304, ring torus shaped body 704 is formed of a substantially rigid material, such as plastic or metal. For example, ring torus shaped body 704 may be formed of an injection molded or milled plastic material and/or a molded, forged, milled and/or otherwise formed metal such as stainless steel, brass, copper, or aluminum metal. Where ring torus shaped body 704 is made at least in part from an electrically conductive metal, ring torus shaped body 704 may have an external insulting coating, such as silicone, rubber, or plastic.

Ring torus shaped body 704 forms a plurality of apertures 712 between internal sensing cavity 706 and exterior 214 to provide a vent area 710 for internal sensing cavity 706 and forms a plurality of apertures 718 between internal sensing cavity 708 and exterior 214 to provide a vent area 716 for internal sensing cavity 708. Advantageously, vent areas 710 and 716 are positioned such that they do not accumulate mucus, food or other debris as animal 104 forages or drinks, as compared to when vent areas a positioned at a lower portion of the ring assembly. Similarly to open-ring assembly 302, methane sensors 220 and 222, control circuit 232 may be positioned within ring torus shaped body 704. In this example, microcontroller 236 may include a power source (e.g., a battery and/or energy harvesters). Square-profile open-ring assembly 702 may include other sensors and functionality without departing from the scope hereof. Square-profile open-ring assembly 702 may include on or more sensors 750(1) and 750(2) for sensing other metrics of animal 104. Sensors 750(1) and 750(2) are positioned at rounded-ends 762(1) and 762(2), respectively, such that sensors 750 are in close proximity of nose 106 when square-profile open-ring assembly 702 is attached to animal 104.

Ring torus shaped body 704 has a removable plate (not shown) that provides access to internal circuitry cavity 730 and is secured by two fasteners that screw into threaded holes 740(1) and 740(2).

### Temperature Sensors

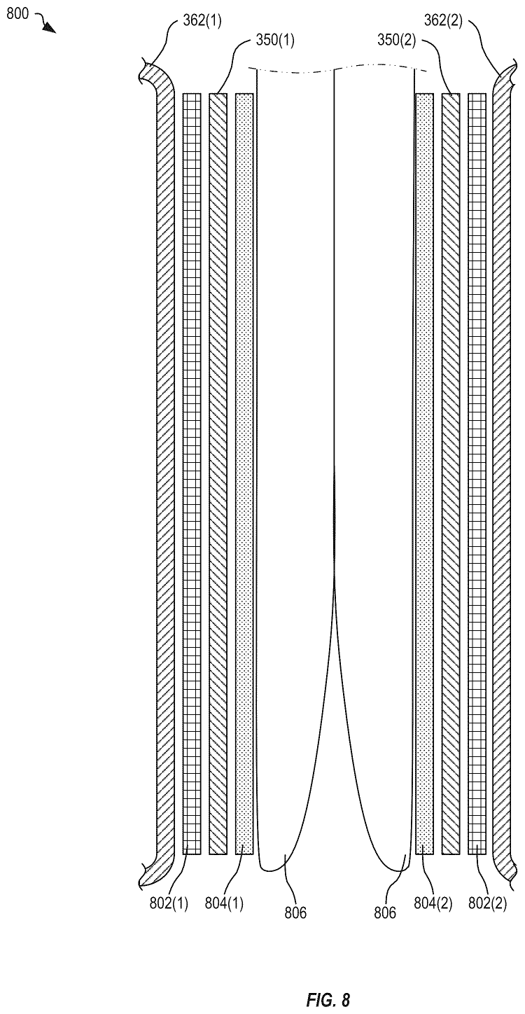

FIG. 8 is a schematic cross-section 800 through open-ring assembly 302 of FIG. 3 illustrating sensors 350 in further example detail, in embodiments. Cross-section 800 may also represent sensors 750 of square-profile open-ring assembly 702 of FIG. 7A.

In this example, sensors 350 are positioned at an external surface of rounded-ends 362. Sensors 350(1) and 350(2) are mounted on flex circuits 802(1) and 802(2), which are attached (e.g., adhered or otherwise fastened) to outer surfaces of rounded-ends 362(1) and 362(2), respectively. For example, sensors 350(1) and 350(2) may connect through structure of ring torus shaped body 304 with control circuit 232 and may be recessed into rounded-ends 362(1) and 362(2). Outer surfaces of sensors 350(1) and 350(2) may be covered by a thermally conductive layer 804(1) and 804(2), respectively, which contact a septum 806 of animal 104 when square-profile open-ring assembly 702 is inserted into nose 106. Thermally conductive layers 804 conduct heat from septum 806 to sensors 350 to ensure an accurate temperature reading.

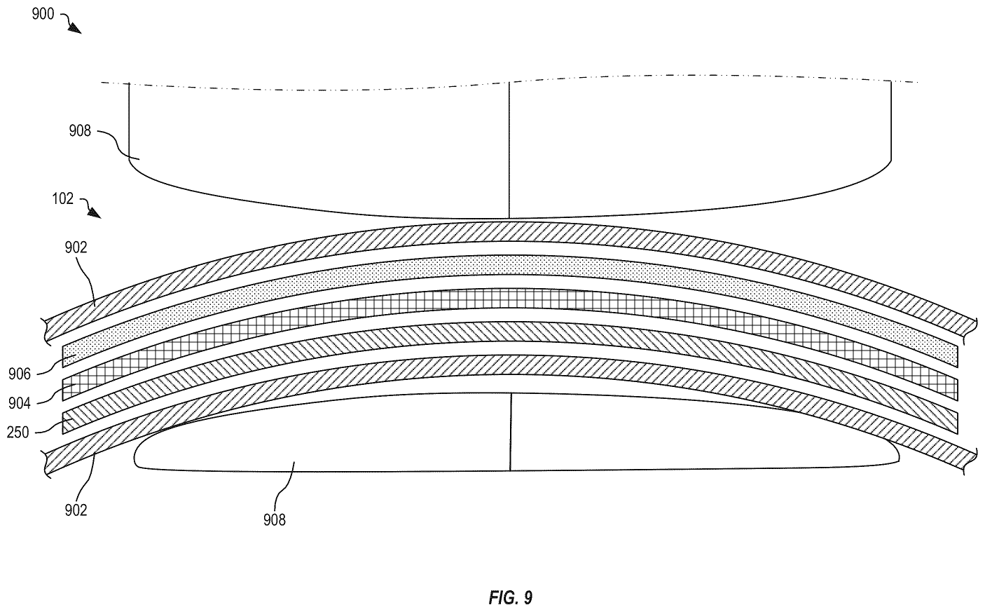

FIG. 9 is a schematic cross-section 900 through closed-ring assembly 202 of FIG. 2 illustrating sensor 250 in further example detail, in embodiments.

In this example, sensor 250 is positioned within a top section of ring torus shaped body 204, as shown in FIG. 2. At the top section, ring torus shaped body 204 is formed of an outer wall 902 in a curved tubular shape. Sensor 250 is mounted on a flex circuit 904, which is attached (e.g., adhered or otherwise fastened) to an inner surface of ring torus shaped body 204 by a thermally conductive layer 906. Outer wall 902 contacts a septum 908 of animal 104 when closed-ring assembly 202 is inserted into nose 106 and through septum 908. Heat from septum 908 is conducted by thermally conductive layer 906 to sensor 250 to ensure an accurate temperature reading.

As appreciated, features of each embodiments may be interchanged and/or combined to form new embodiments without departing from the scope hereof.

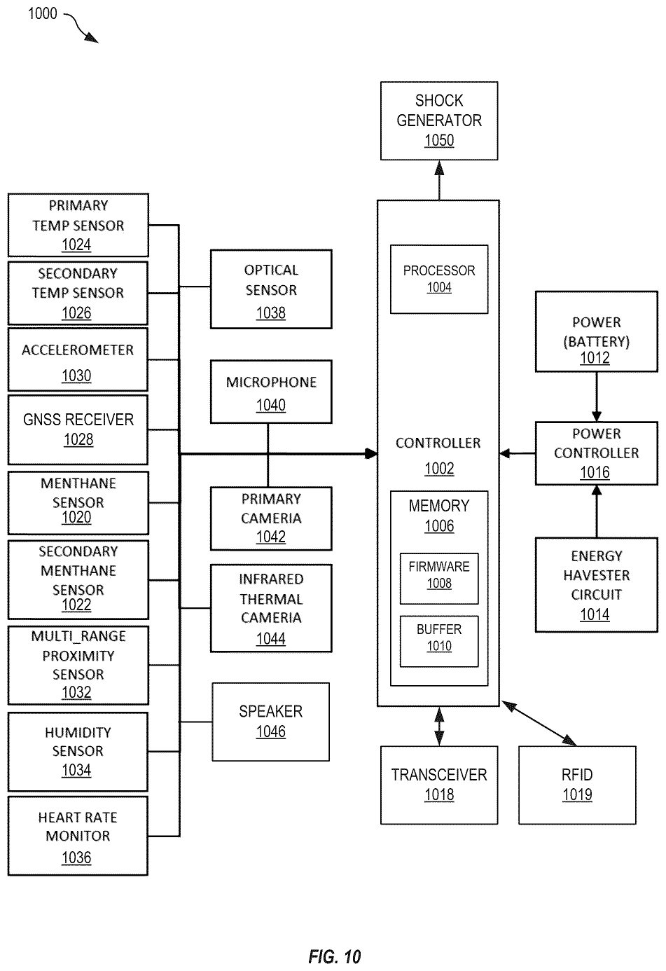

FIG. 10 is a block diagram illustrating example circuitry 1000 that may be used with any of ring assemblies 102/202/302/502/602/702 of FIGS. 1, 2, 3, 5A-5E, 6A-6D, and 7A, respectively, (hereinafter referenced collectively as ring assembly 102) in embodiments. That is, components of circuitry 1000 may be used to implement any of the embodiments described above and shown in FIGS. 1, 2, 3, 5A-5E, 6A-6D, and 7A.

Circuitry 1000 includes a microcontroller 1002 with at least one processor 1004 and memory 1006 storing firmware 1008 and a data buffer 1010. Circuitry 1000 is powered from a power source 1012 (e.g., a battery) that may be charged from an energy harvester circuit 1014 (e.g., solar collector, kinetic energy converter, electromagnetic charging, etc.). A power controller 1016 operates to recharge power source 1012 and supply conditioned power to other components of circuitry 1000. Firmware 1008 includes machine-readable instructions that when executed by at least one processor 1004 cause microcontroller 1002 to implement functionality of ring assembly 102 as described herein. Data buffer 1010 may be used to store methane data (e.g., methane data 424 of FIG. 4) prior to local processing and/or transmission to data service 110. Circuitry 1000 may also include a transceiver 1018 (e.g., transceiver 238) that transmits sensed data (e.g., methane data) to data service 110.

Circuitry 1000 includes at least one methane sensor 1020/1022 (e.g., methane sensors 220/222 of FIGS. 2-4) for sensing a level of methane in air. Circuitry 1000 also includes at least one temperature sensor 1024/1026 (e.g., sensor 250/252/350/352) for sensing a temperature of animal 104 and/or an ambient temperature. Circuitry 1000 may also include a GNSS receiver 1028 (e.g., GNSS receiver 240) for determining a geographic location of ring assembly 102. Circuitry 1000 is also shown with other optional sensors that may be included within circuitry 1000 to capture certain data for various use scenarios of ring assembly 102. For example, circuitry 1000 may include at least one accelerometer 1030 (e.g., multi-axis x, y, z) for sensing movement of animal 104, a proximity sensor 1032 for sensing proximity of circuitry 1000 (e.g., closed-ring assembly 202, open-ring assembly 302, square-profile open-ring assembly 702, etc.) to other ring assemblies based on detected wireless signals, a humidity sensor 1034 for sensing an ambient humidity level, a heart-rate monitor 1036 for sensing a heart-rate of animal 104, an optical sensor 1038 for sensing an ambient light level (e.g., day, light), a microphone 1040 for sensing ambient sounds and or sounds of animal 104, a camera 1042 for capturing optical images, an infrared thermal camera 1044 for capturing thermal images, and a speaker 1046 for generating an audible sound (e.g., a tone or alarm). Circuitry 1000 may include other sensors without departing from the scope hereof. For example, circuitry 1000 may also include an oxygen saturation monitor and a blood flow sensor. Advantageously, circuitry 1000 monitors an animal to which it is attached, and thereby determines a status of the animal.

In certain embodiments, circuitry 1000 is configured to receive, via transceiver 1018, data from one or more auxiliary sensors. For example, an auxiliary sensor may be located proximate animal 104 (e.g., separate from ring assembly 102) and configured to transmit auxiliary parametric data to ring assembly 102, whereby ring assembly 102 is further programmed to relay the auxiliary parametric data to data service 110 and/or used the auxiliary parametric data within algorithms implemented by firmware 1008.

In certain embodiments, circuitry 1000 also includes a radio frequency identification (RFID) transponder 1019 that allows a unique ID of ring assembly 102 to be read using an RFID reader. In certain embodiments, RFID transponder 1019 is controlled by circuitry 1000. Further, reporting of methane data and other sensed metrics may be triggered by the RFID transponder.

Ring assembly 102 may include any combination of components described for circuitry 1000. Microcontroller 1002 may be implemented by any type of embedded microcontroller and may implement certain of the described sensors. For example, microcontroller 1002 may include one or more interfaces (e.g., digital, analog-ADC-etc.) for communicating with, or capturing data from, the included sensors. Circuitry 1000 may be fabricated on a flex circuit that is sized and shaped to fit within internal cavities of ring assembly 102 to position sensors at required positions.

In certain embodiments, ring assembly 102 facilitates calculating and forecasting animal health of animal 104. For example, ring assembly 102 may sense a temperature of the septum of animal 104 and/or other parametric data of the animal. In certain embodiments, firmware 1008 may adjust a sensed temperature value of animal 104 based on a sensed ambient temperature, whereby the adjustment estimates a core temperature of the animal. In other embodiments, software 408 of data service 110 estimates the core temperature of animal 104 based on received data from ring assembly 102.

One or both of software 408 and firmware 1008 may determine a health state of animal 104 based on localized changes in sensed temperatures over time and threshold values. For example, rapid changes in sensed temperatures may indicate a stressed state of animal 104.

In embodiments where 104 includes one or both of at least one accelerometer 1030 and GNSS receiver 1028, circuitry 1000 may track a an activity level and motion of animal 104.

In certain embodiments where circuitry 1000 includes GNSS receiver 1028, circuitry 1000 may also include a shock generator 1050 for generating an electric shock (e.g., an electrical pulse). Memory 1006 may be configured with a geofenced area (e.g., geographic data defining a boundary of an area containing animal 104) whereby firmware 1008 compares a current location reported by GNSS receiver 1028 against the geofenced area to determine whether the animal has crossed the geofence. Ring assembly 102 may further include at least one electrode at the top of ring torus shaped body 204 or at rounded-ends 362 of ring torus shaped body 304 to contact nose 106 and/or the septum of animal 104, whereby circuitry 1000 controls shock generator 1050 to generate an electrical voltage (e.g., an electric shock) at the electrodes when the animal approaches or crosses the geofence. In embodiments where ring torus shaped body 204 is electrically conducting (e.g., metal), ring torus shaped body 204 forms a first electrode (e.g., connected as a ground of control circuit 232) and a second electrode is positioned at a top portion of ring torus shaped body 204 such that it contacts nose 106 of animal 104. For open-ring assembly 302, one electrode is positioned at rounded-end 362(1) and a second electrode is positioned at rounded-end 362(2), and both electrodes are in contact with nose 106 of animal 104. One electrode is grounded and the other electrode provides the electric shock to the animal. In certain embodiments, circuitry 1000 is configured to receive, via transceiver 1018, the geographic area defining the boundary, whereby circuitry 1000 stores the boundary within memory 1006.

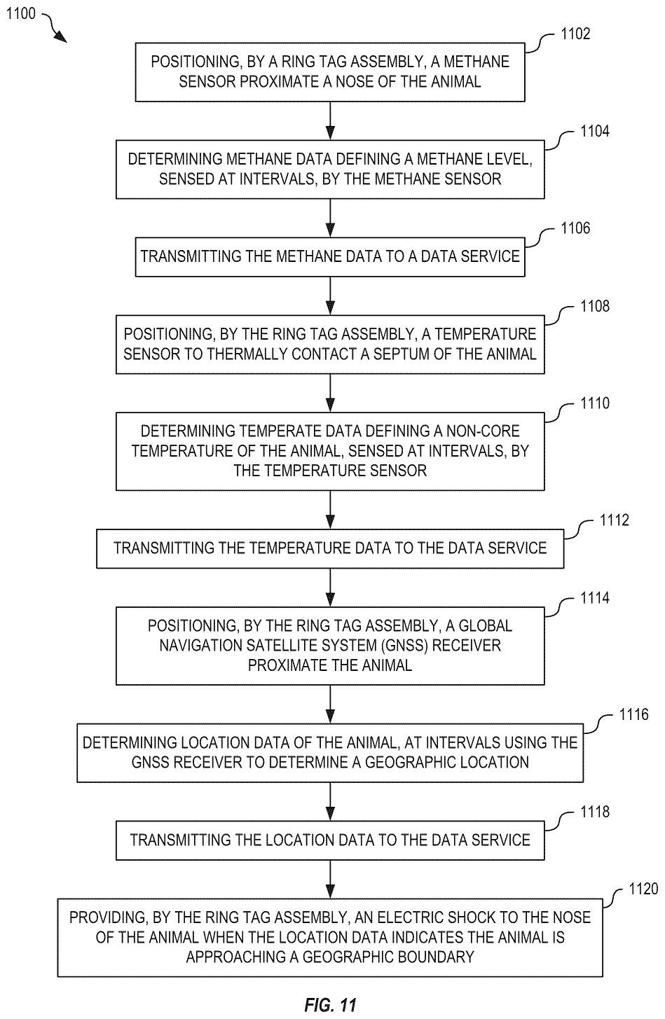

FIG. 11 is a flowchart illustrating one example method 1100 implemented by ring assembly 102 of FIG. 1, in embodiments. Method 1100 is implemented at least in part by a physical structure of ring assembly 102 and at least in part by control circuit 232 of FIG. 2, for example.

In block 1102, method 1100 positions, by a ring assembly, a methane sensor proximate a nose of the animal. In one example of block 1102, ring assembly 202/302/702 positions methane sensors 220/222 proximate nose 106 to sense enteric fermentation methane in breath of animal 104. In block 1104, method 1100 determines methane data defining a methane level, sensed at intervals, by the methane sensor. In one example of block 1104, control circuit 232 determines methane data 424 by sampling methane sensors 220/222 at intervals. In block 1106, method 1100 transmits the methane data to a data service. In one example of block 1106, microcontroller 236 controls transceiver 238 to transmit methane data 424 to data service 110 at intervals.

Blocks 1108 through 1112 are optional. In block 1108, method 1100 positions, by the ring assembly, a temperature sensor to thermally contact a septum of the animal. In one example of block 1108, ring assembly 202/302/702 positions sensor 250 at a top of ring torus shaped body 204 to contact a septum of animal 104. In block 1110, method 1100 determines temperate data defining a non-cote temperature of the animal, sensed at intervals, by the temperature sensor. In one example of block 1110, control circuit 232 controls sensor 250 to sense a temperature of animal 104 at intervals. In block 1112, method 1100 transmits the temperature data to the data service. In one example of block 1112, microcontroller 236 controls transceiver 238 to send temperature data from sensor 250 to data service 110.

Blocks 1114 through 1120 are optional. In block 1114, method 1100 positions, by the ring assembly, a global navigation satellite system (GNSS) receiver proximate the animal. In one example of block 1114, ring assembly 202/302/702 positions GNSS receiver 240 near animal 104. In block 1116, method 1100 determines location data of the animal, at intervals using the GNSS receiver to determine a geographic location. In one example of block 1116, microcontroller 236 controls GNSS receiver 240 to determine a current location of animal 104. In block 1118, method 1100 transmitting the location data to the data service. In one example of block 1118, microcontroller 236 controls transceiver 238 to send the geographic location data determined from GNSS receiver 240 to data service 110.

In block 1120 method 1100 provides, by the ring assembly, an electric shock to the nose of the animal when the location data indicates the animal is approaching a geographic boundary. In one example of block 1120, microcontroller 1002 controls shock generator 1050 to provide an electrical shock to nose 106 when a current location determined by GNSS receiver 240 indicates animal 104 is approaching a geographic boundary defined within memory 1006 of microcontroller 1002.

In certain embodiments, ring assembly 102 further includes a light emitting diode (LED) the is controlled by firmware 1008 to provide a visual indication of biometric and/or behavior and/or location changes of the animal.

### Network

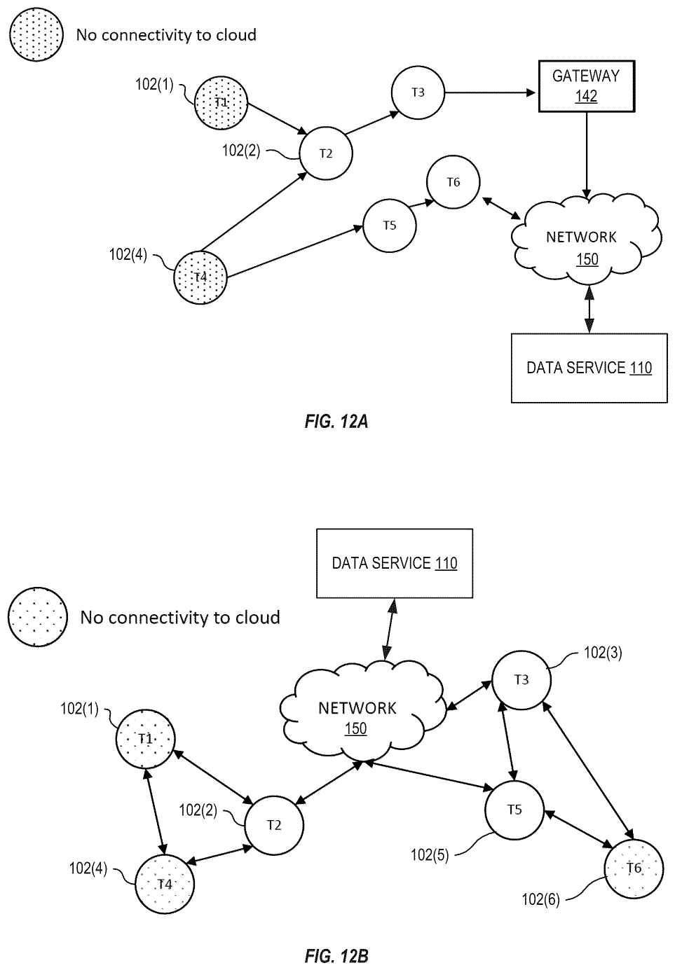

FIGS. 12A and 12B illustrate example wireless connectivity of ring assembly 102 of FIG. 1 with network 150, in embodiments. Ring assembly 102 represents any of ring assemblies 202/302/502/602/702 of FIGS. 1, 2, 3, 5A-5E, 6A-6D, and 7A that send methane data to data service 110 via communication network 150 (e.g., any one or more protocols including Bluetooth, Wi-Fi, ANT, LoRa, Internet, cellular, and satellite).

FIG. 12A illustrates one example scenario where a livestock herd fitted with ring assemblies 102(1)-102(6) are roaming in a field and ring assemblies 102(1) and 102(4) are unable to communicate directly with communication network 150 (e.g., when ring assemblies 102(1) and 102(4) are out of wireless range of relay device 142 and do not include cellular or satellite protocols). In this scenario, methane data (and other sensor data) wireless transmissions sent by ring assemblies 102(1) and 102(4) are received and retransmitted (e.g., relayed) by at least one other ring assembly 102(2), 102(3), 102(5) and 102(6) such that they are received by data service 110 via at least one relay device 142 and or via communication network 150. For example, ring assemblies 102(1)-(6) may form a mesh network capable of relaying methane data to data service 110 and/or of relaying wireless messages to an individual ring assembly 102.

FIG. 12B illustrates another example scenario where a livestock herd fitted with ring assemblies 102(1)-102(6) are roaming in a field that does not include relay device 142 and ring assemblies 102(1) and 102(4) are unable to communicate directly with communication network 150 since they do not include cellular or satellite protocols. In this scenario, methane data (and other sensor data) wireless transmissions sent by ring assemblies 102(1) and 102(4) are received and retransmitted (e.g., relayed) by at least one other ring assembly 102(2), 102(3), 102(5) and 102(6) such that they are received by data service 110 via at least one relay device 142 and or via communication network 150. For example, ring assemblies 102(1)-(6) may form a mesh network where at least one ring assembly 102 is capable of relaying methane data to data service 110 and/or of relaying wireless messages to an individual ring assembly 102.

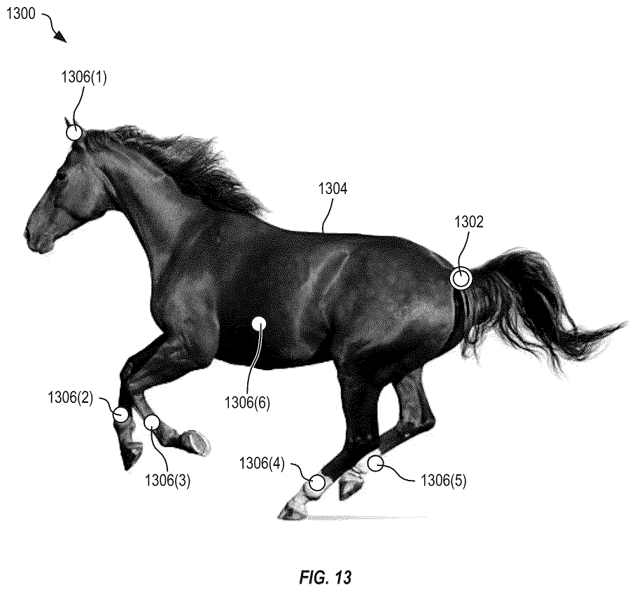

FIG. 13 is an image 1300 showing one example ring assembly 1302 fitted to a tail of an animal 1304, in embodiments. In this example, animal 1304 is a horse but ring assembly 1302 may be similarly attached to other animals without departing from the scope hereof. Ring assembly 1302 is similar to closed-ring assembly 202 of FIG. 2 and includes similar functionality. In this embodiments, ring assembly 1302 is configured with a friction coating (e.g., rubber) that facilitates attachment and retention of the ring assemble at a base of a tail of animal 1304, wherein ring assembly 1302 detects levels of methane gas expelled by animal 1304. Ring assembly 1302 may also receive data (e.g., as short-range wireless signals such as Bluetooth) from one or more additional sensor units 1306 attached to animal 1304. Sensor units 1306 may not include methane (or other gas) detection, but may include one or more other sensors, such as a multi-axis accelerometer, a light sensors, EKG sensors, and temperature sensos. Accordingly, ring assembly 1302 may collect addition data of animal 1304 from additional sensor units 1306 attached to animal 1304. As shown in FIG. 13, sensor unit 1306(1) is attached to an car of animal 1304; sensor unit 1306(2)-(5) are attached to different legs of animal 1304; and sensor unit 1306(6) is attached to a body of animal 1304, each sensor unit 1306 includes a short range transceiver (e.g., Bluetooth) for communicating with ring assembly 1302 and may not include long range wireless capability.

Ring assembly 1302 collects data from internal sensors (e.g., as described above for closed-ring assembly 202) and receives additional data via short range wireless communications from external sensor units 1306. This additional data may include one or more of accelerometer data, ambient light data, EKG data, and temperature data, from different parts of animal 1304. Similarly to closed-ring assembly 202, ring assembly 1302 may send the collected data to data service 110 using a long range wireless protocol, such as cellular and/or satellite, or via a short range wireless protocol and a relay device (e.g., Bluetooth protocol to relay device 142 of FIG. 1).

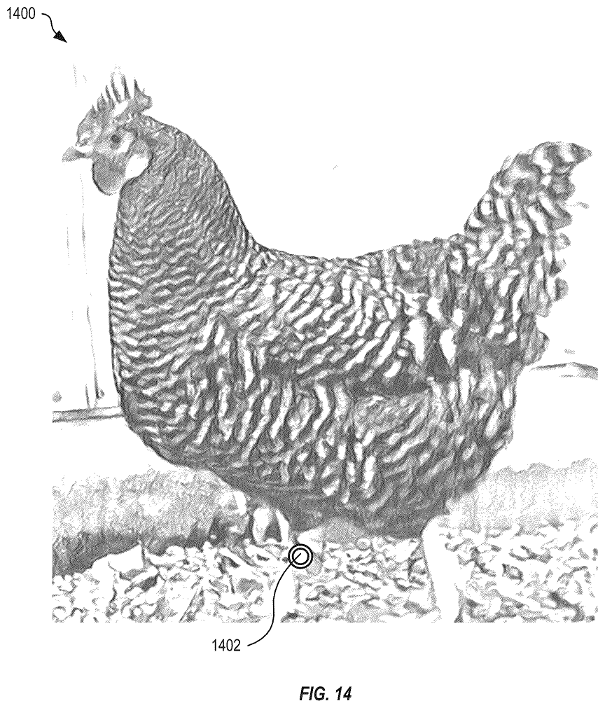

FIG. 14 is an image 1400 showing one example ring assembly 1402 attached to a leg of a bird 1404, in embodiments. In this example, bird 1404 is a chicken; however, ring assembly 1402 may be attached to a leg of other birds without departing from the scope hereof. Ring assembly 1402 is similar to closed-ring assembly 202 of FIG. 2, but may not include a methane sensor. For example, one example ring assembly 1402 may include a temperature sensor, a light sensor, and a multi-axis accelerometer. Similarly to closed-ring assembly 202, ring assembly 1302 may send the collected data to data service 110 using a long range wireless protocol, such as cellular and/or satellite, or via a short range wireless protocol (e.g., Bluetooth) and a relay device (e.g., relay device 142 of FIG. 1).

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### Combination of Features

Features described above as well as those claimed below may be combined in various ways without departing from the scope hereof. The following enumerated examples illustrate some possible, non-limiting combinations:

(A1). A ring assembly for measurement of methane from an animal, including: a ring torus shaped body forming an internal sensing cavity having a vent area between the internal cavity and an exterior of the ring torus shaped body; a methane sensor positioned at the internal cavity for sensing a methane level in gas entering the internal sensing cavity via the vent area; a control circuit, positioned within an internal circuitry cavity formed by the ring torus shaped body, electrically coupled with the methane sensor, the control circuit having: a transceiver; and a microcontroller programmed to: read, at intervals, a methane measurement from the methane sensor to form methane data; and send the methane data via the transceiver to a data service; and a power source positioned within the internal circuitry cavity for providing power to the control circuit.

(A2). In the embodiment (A1), the ring torus shaped body being formed of a substantially rigid material.

(A3). In either of embodiments (A1) or (A2), the ring torus shaped body having an external coating of electrically insulating material.

(A4). In any of the embodiment (A1)-(A3), the internal circuitry cavity being fluidly isolated from the internal sensing cavity.

(A5). In any of the embodiment (A1)-(A4), the vent area being positioned to capture enteric emissions in breath of the animal when the ring assembly is attached to a nose of the animal.

(A6). In any of the embodiment (A1)-(A5), the ring torus shaped body having a hinge and a clasp, wherein the clasp is opened and the ring torus shaped body is positioned through a septum of the animal.

(A7). In any of the embodiment (A1)-(A6), the ring torus shaped body forming a gap, the ring assembly further comprising: a first rounded-end formed at a first end of the ring torus shaped body; and a second rounded-end formed at a second end of the ring torus shaped body; wherein the ring assembly is attached to a nose of the animal by positioning the first rounded-end and the second rounded-end either side of a septum of the animal.

(A8). In any of the embodiment (A1)-(A7), the first rounded-end and the second rounded-end being one of a ball-end, a rounded square-end, and a rounded disc-end.

(A9). In any of the embodiment (A1)-(A8), the microcontroller being further programmed to process the methane data prior to sending via the transceiver.

(A10). In any of the embodiment (A1)-(A9), the data service being a cloud-based service that processes the methane data.

(A11). In any of the embodiment (A1)-(A10), the methane data being stored by the microcontroller and sent via the transceiver in batches.

(A12). In any of the embodiment (A1)-(A11), the methane data defining methane levels in enteric emissions from a mouth and a nose of the animal as a single data point.

(A13). In any of the embodiment (A1)-(A12), the methane data defining methane levels in enteric emissions from a mouth and a nose of the animal over a selected time interval.

(A14). In any of the embodiment (A1)-(A13), the time interval being selected from the group consisting of one hour, one day, one week.

(A15). In any of the embodiment (A1)-(A14), the methane data defining a baseline measurement for the animal.

(A16). In any of the embodiment (A1)-(A15), the microcontroller being further programmed to process the methane data to determine a change in magnitude in the methane measurement relative to baseline methane measurement determined for the animal.

(A17). In any of the embodiment (A1)-(A16), wherein the methane measurements of the animal are determined to be less or more as a result of new inputs such as feed, location, and stage of life.

(A18). In any of the embodiment (A1)-(A17), further comprising a second methane sensor positioned in a second internal sensing cavity having a second vent area between the second internal cavity and the exterior, the microcontroller being further programmed to read, at intervals, a second methane measurement from the second methane sensor to form methane data, wherein the control circuit further operates to determine methane levels in enteric emissions from a mouth and a nose of the animal.

(A19). In any of the embodiment (A1)-(A18), further comprising determining a health state of the animal based on a difference between a septum temperature data and an ambient temperature.

(A20). In any of the embodiment (A1)-(A19), further comprising at least one additional sensor senses additional parametric data of the animal, the control circuit sensing the additional parametric data to the data service via the transceiver.

(A21). In any of the embodiment (A1)-(A20), the at least one additional sensor being selected from the group comprising an activity monitor, a global navigation satellite system (GNSS) receiver, an optical sensor, a humidity sensor, a light sensor, a proximity sensor, a heart-rate monitor, an oxygen saturation monitor, a blood flow sensor, a core body temperature sensor, and a multi-axis accelerometer.

(A22). In any of the embodiment (A1)-(A21), further comprising a shock generator for providing an electric shock to a nose of the animal when a geographic location determined by the GNSS receiver indicate the animal is approaching a geographic boundary.

(A23). In any of the embodiment (A1)-(A22), the shock generator generating an electrical voltage between metal of the ring torus shaped body and an electrode positioned at a top portion of the ring torus shaped body to contact the nose.

(B1). A method for determining enteric fermentation methane in breath of an animal, including: positioning, by a ring assembly, a methane sensor proximate a nose of the animal; determining methane data defining a methane level, sensed at intervals, by the methane sensor; and sending the methane data to a data service.

(B2). In the embodiment (B1), the ring assembly physically coupling with a septum of the animal.

(B3). Either of the embodiments (B1) or (B2) further including: positioning, by the ring assembly, a temperature sensor proximate the nose; determining temperature data defining a non-core temperature of the animal, sensed at intervals by the temperature sensor; and transmitting the temperature data to the data service.

(B4). Any of the embodiment (B1)-(B3) further including: positioning, by the ring assembly, a global navigation satellite system (GNSS) receiver proximate the animal; determining location data of the animal, at intervals using the GNSS receiver to determine a geographic location; and transmitting the location data to the data service.

(B5). Any of the embodiment (B1)-(B3) further including providing, by the ring assembly, an electric shock to the nose of the animal when the location data indicates the animal is approaching a geographic boundary defined within the ring assembly.

(C1). A ring assembly for monitoring an animal, including: a ring torus shaped body forming at least one internal cavity; at least one sensor positioned in the at least one cavity for sensing a status of the animal; a control circuit, positioned within the at least one cavity, electrically coupled with the at least one sensor and having: a transceiver; and a microcontroller programmed to: read, at intervals, a sensor data from the at least one sensor; and send the sensor data via the transceiver to a data service; and a power source positioned within the at least one cavity for providing power to the control circuit.

(C2). In the embodiment (C1), the ring torus shaped body including a friction coating to retaining the ring assembly at a based of a tail of the animal.

(C3). In either of the embodiments (C1) or (C2), the transceiver being configured to receive sensor data from at least one additional sensor unit attached to the animal.

(C4). In any of the embodiments (C1)-(C3), the animal being a bird and the ring assembly attaching to a leg of the bird.

What is claimed is:

1. A ring assembly for measurement of methane from an animal, comprising:
a ring torus shaped body forming an internal sensing cavity having a vent area between the internal cavity and an exterior of the ring torus shaped body;
a methane sensor positioned at the internal cavity for sensing a methane level in gas entering the internal sensing cavity via the vent area;
a control circuit, positioned within an internal circuitry cavity formed by the ring torus shaped body, electrically coupled with the methane sensor, the control circuit having:
a transceiver; and
a microcontroller programmed to:
read, at intervals, a methane measurement from the methane sensor to form methane data; and
send the methane data via the transceiver to a data service; and
a power source positioned within the internal circuitry cavity for providing power to the control circuit.

2. The ring assembly of claim 1, the ring torus shaped body being formed of a substantially rigid material.

3. The ring assembly of claim 1, the ring torus shaped body having an external coating of electrically insulating material.

4. The ring assembly of claim 1, the internal circuitry cavity being fluidly isolated from the internal sensing cavity.

5. The ring assembly of claim 1, the vent area being positioned to capture enteric emissions in breath of the animal when the ring assembly is attached to a nose of the animal.

6. The ring assembly of claim 1, the ring torus shaped body having a hinge and a clasp, wherein the clasp is opened and the ring torus shaped body is positioned through a septum of the animal.

7. The ring assembly of claim 1, the ring torus shaped body forming a gap, the ring assembly further comprising:
a first rounded-end formed at a first end of the ring torus shaped body; and
a second rounded-end formed at a second end of the ring torus shaped body;
wherein the ring assembly is attached to a nose of the animal by positioning the first rounded-end and the second rounded-end either side of a septum of the animal.

8. The ring assembly of claim 7, the first rounded-end and the second rounded-end being one of a ball-end, a rounded square-end, and a rounded disc-end.

9. The ring assembly of claim 1, the microcontroller being further programmed to process the methane data prior to sending via the transceiver.

10. The ring assembly of claim 1, the data service being a cloud-based service that processes the methane data.

11. The ring assembly of claim 1, the methane data being stored by the microcontroller and sent via the transceiver in batches.

12. The ring assembly of claim 1, the methane data defining methane levels in enteric emissions from a mouth and a nose of the animal as a single data point.

13. The ring assembly of claim 1, the methane data defining methane levels in enteric emissions from a mouth and a nose of the animal over a selected time interval.

14. The ring assembly of claim 13, the time interval being selected from the group consisting of one hour, one day, one week.

15. The ring assembly of claim 1, the methane data defining a baseline measurement for the animal.

16. The ring assembly of claim 1, the microcontroller being further programmed to process the methane data to determine a change in magnitude in the methane measurement relative to baseline methane measurement determined for the animal.

17. The ring assembly of claim 1, wherein the methane measurements of the animal are determined to be less or more as a result of new inputs such as feed, location, and stage of life.

18. The ring assembly of claim 1, further comprising a second methane sensor positioned in a second internal sensing cavity having a second vent area between the second internal cavity and the exterior, the microcontroller being further programmed to read, at intervals, a second methane measurement from the second methane sensor to form methane data, wherein the control circuit further operates to determine methane levels in enteric emissions from a mouth and a nose of the animal.

19. The ring assembly of claim 1, further comprising determining a health state of the animal based on a difference between a septum temperature data and an ambient temperature.

20. The ring assembly of claim 1, further comprising at least one additional sensor senses additional parametric data of the animal, the control circuit sensing the additional parametric data to the data service via the transceiver.

21. The ring assembly of claim 20, the at least one additional sensor being selected from the group comprising an activity monitor, a global navigation satellite system (GNSS) receiver, an optical sensor, a humidity sensor, a light sensor, a proximity sensor, a heart-rate monitor, an oxygen saturation monitor, a blood flow sensor, a core body temperature sensor, and a multi-axis accelerometer.

22. The ring assembly of claim 21, further comprising a shock generator for providing an electric shock to a nose of the animal when a geographic location determined by the GNSS receiver indicate the animal is approaching a geographic boundary.

23. The ring assembly of claim 22, the shock generator generating an electrical voltage between metal of the ring torus shaped body and an electrode positioned at a top portion of the ring torus shaped body to contact the nose.

24. A method for determining enteric fermentation methane in breath of an animal, comprising:
positioning, by a ring assembly, a methane sensor proximate a nose of the animal;
determining methane data defining a methane level, sensed at intervals, by the methane sensor; and
sending the methane data to a data service.

25. The method of claim 24, wherein the ring assembly physically couples with a septum of the animal.

26. The method of claim 24, further comprising:
positioning, by the ring assembly, a temperature sensor proximate the nose;
determining temperature data defining a non-core temperature of the animal, sensed at intervals by the temperature sensor; and
transmitting the temperature data to the data service.

27. The method of claim 24, further comprising:
positioning, by the ring assembly, a global navigation satellite system (GNSS) receiver proximate the animal;
determining location data of the animal, at intervals using the GNSS receiver to determine a geographic location; and
transmitting the location data to the data service.

28. The method of claim 27, further comprising providing, by the ring assembly, an electric shock to the nose of the animal when the location data indicates the animal is approaching a geographic boundary defined within the ring assembly.

29. A ring assembly for monitoring an animal, comprising:
a ring torus shaped body forming at least one internal cavity;
at least one sensor positioned in the at least one cavity for sensing a status of the animal;
a control circuit, positioned within the at least one cavity, electrically coupled with the at least one sensor and having:
a transceiver; and
a microcontroller programmed to:
read, at intervals, a sensor data from the at least one sensor; and
send the sensor data via the transceiver to a data service; and
a power source positioned within the at least one cavity for providing power to the control circuit.

30. The ring assembly of claim 29, the ring torus shaped body comprising a friction coating to retaining the ring assembly at a based of a tail of the animal.

31. The ring assembly of claim 29, the transceiver being configured to receive sensor data from at least one additional sensor unit attached to the animal.

32. The ring assembly of claim 29, the animal being a bird and the ring assembly attaching to a leg of the bird.
